(19) United States
(12) Patent Application Publication
(10) Pub. No.: US 2012/0238846 A1
(43) Pub. Date: Sep. 20, 2012

(54) VASCULAR OCCLUSION TEST APPARATUS, SYSTEMS, AND METHODS FOR ANALYZING TISSUE OXYGENATION

(75) Inventors: Dean E. Myers, Stewart, MN (US); Roger W. Schmitz, Hutchinson, MN (US); Kevin Becker, Glencoe, MN (US)

(73) Assignee: Hutchinson Technology Incorporated, Hutchinson, MN (US)

(21) Appl. No.: 13/051,140

(22) Filed: Mar. 18, 2011

(51) Int. Cl. A61B 5/1455

(52) U.S. Cl. 600/324

(57) ABSTRACT

A vascular occlusion test apparatus, systems, and methods for analyzing tissue oxygen saturation levels in patients are disclosed. A system for analyzing data related to tissue oxygenation in a patient includes a blood pressure device, a tissue oxygen sensor, and a control module in communication with the blood pressure device and tissue oxygen sensor. The control module includes a processor that computes various tissue characteristics associated with tissue oxygenation, including ischemia slope and recovery slope. During a vascular occlusion test, the control module can be configured to control an inflatable cuff based on tissue oxygen measurements obtained from the tissue oxygen sensor.

### TECHNICAL FIELD

The present disclosure relates generally to analyzing tissue oxygenation. More specifically, the present disclosure pertains to a vascular occlusion test apparatus, systems, and methods for analyzing tissue oxygen saturation (StO₂) levels in patients.

### BACKGROUND

Tissue oxygenation may be analyzed as a means of monitoring and diagnosing shock, sepsis, and other types of diseases as well as monitoring a patient's overall health. Typically, tissue oxygenation is monitored by either measuring hemoglobin oxygen saturation in the blood or, alternatively, by measuring transcutaneous partial pressure of oxygen. Hemoglobin oxygen saturation (SO₂, SaO₂, SpO₂) is typically expressed as a percent, and represents the oxygen present on the hemoglobin in circulating blood divided by the total possible oxygen that could be carried by hemoglobin. Transcutaneous partial pressure of oxygen (PO₂) measures the amount of oxygen drawn to the skin's surface by a heated sensor, and provides an estimate of arterial partial pressure of oxygen.

StO₂ is the quantification of the ratio of oxygenated hemoglobin to total hemoglobin in the microcirculation of skeletal muscle, and is an absolute number. In some cases, the measurement of StO₂ is taken with a noninvasive, fiber-optic light that illuminates tissue below the level of the skin. An example technique for illuminating tissue below the surface of the skin is known as near infrared spectroscopy (NIRS), which uses specific, calibrated wavelengths of near infrared light to noninvasively illuminate the tissue below the skin surface. These wavelengths of light scatter in the tissue and are absorbed differently depending on the amount of oxygen attached to hemoglobin in the arterioles, venules, and capillaries. Light that is not absorbed is returned as an optical signal and is analyzed to produce a ratio of oxygenated hemoglobin to total hemoglobin, expressed as % StO₂. In practice, near infrared light penetrates tissue such as skin, bone, muscle and soft tissue where it is absorbed by chromophores such as hemoglobin and myoglobin that have absorption wavelengths in the near infrared region (i.e., approximately 700-1000 nm). These chromophores vary in their absorbance of NIRS light, depending on changes in the oxygenation state of the tissue. Complex algorithms differentiate the absorbance contribution of the individual chromophores.

Vascular occlusion test (VOT) devices that rely on the absorbance of NIRS light during and after an induced ischemic event have been introduced for measuring tissue oxygen consumption and microvascular reperfusion and reactivity. In some procedures, a separate blood pressure device is used in conjunction with the VOT device in order to measure systolic blood pressure immediately preceding a VOT test in order to identify a target tourniquet pressure needed to stop blood flow and induce ischemia. In some cases, for example, the blood pressure device comprises a sphygmomanometer with an inflatable blood pressure cuff that is placed around a limb of the patient (e.g., an arm or leg) and inflated at a time before the VOT device is tasked to take StO₂ measurements.

Blood pressure readings obtained from the blood pressure device are not always representative of the actual blood pressure at the measurement site where the VOT testing is to occur. In some cases, inaccuracies can result from variability in the particular cuff design, the placement location of the cuff relative to the VOT device, the patient's posture or orientation, as well as other factors. In some cases, the difference between the sensed blood pressure values and the actual blood pressure values immediately before the VOT test is to begin can result in the VOT device applying an insufficient amount of inflation pressure to the patient's limb for stopping blood flow. As a result, the VOT device may not be able to establish the proper conditions for inducing ischemia at the measurement site, which can cause inaccuracies in the StO₂ measurements at different points throughout the VOT test.

### SUMMARY

The present invention pertains to a vascular occlusion test (VOT) apparatus, systems, and methods for analyzing tissue oxygen saturation (StO₂) levels in patients. A system for analyzing data related to tissue oxygenation in a patient comprises a blood pressure device including a blood pressure sensor and a means for restricting blood flow to an arm or limb of the patient, a tissue oxygen sensor configured to gather data on a tissue chromophore whose light properties depend on the oxygenated state of tissue, and a control module in communication with the blood pressure device and tissue oxygen sensor. In some embodiments, the control module is configured to control the operation of the restriction means based at least in part on one or more measurements sensed by the tissue oxygen sensor. A user interface such as a remote touch screen can be used to display, and in some embodiments store, blood pressure measurements and tissue oxygen measurements obtained during a vascular occlusion test.

An example method for analyzing data related to tissue oxygenation in a patient comprises activating a means for restricting blood flow to an arm or limb of a patient, determining a target tourniquet pressure for inducing ischemia within the arm or limb, obtaining a number of baseline tissue oxygen measurements from the patient while the restriction means is in an unrestricted state, determining a baseline average StO₂ value from the baseline tissue oxygen measurements, controlling the restriction means to a pressure at or near the target tourniquet pressure during a first period of time, determining an ischemia slope start time and an ischemia slope end time during the first period of time, determining an ischemia slope between the ischemia slope start time and the ischemia slope end time, controlling the operation of the restriction means to un-restrict blood flow to the arm or limb during a second period of time, determining a recovery slope start time and a recovery slope end time during the second period of time, determining a recovery slope between the recovery slope start time and the recovery slope end time, and storing one or more tissue oxygen measurements in a memory unit.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagrammatic view showing an example system for gathering, analyzing, and displaying data related to tissue oxygenation;

FIG. 2 is a schematic view showing several illustrative components of the blood pressure device, tissue oxygenation sensor, and control module of FIG. 1;

FIG. 3 is a block diagram showing an example process for configuring the blood pressure device, tissue oxygen sensor, and control module of FIGS. 1-2 for use with a particular patient for taking tissue oxygen measurements;

FIGS. 4A-4C is a block diagram showing an example process for obtaining one or more tissue oxygen measurements from a patient using the system of FIG. 1;

FIG. 5 is a block diagram showing an example process for obtaining a target tourniquet pressure reading using the system of FIG. 1; and

FIG. 6 is a block diagram showing an example process for determining a baseline average value using the system of FIG. 1.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

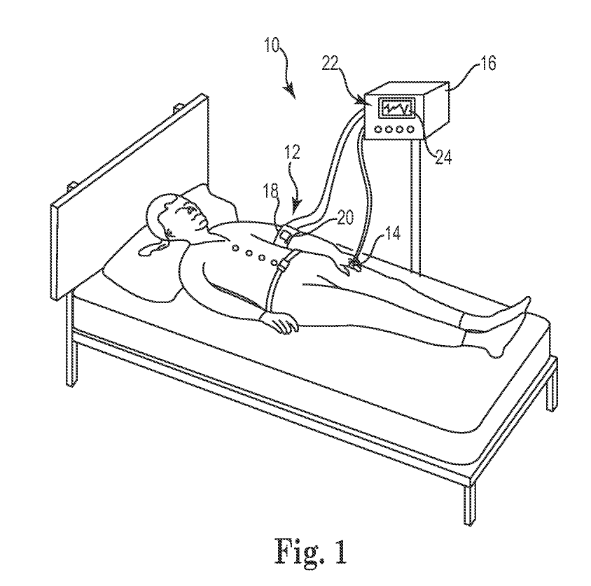

FIG. 1 is a diagrammatic view showing an illustrative system 10 for gathering, analyzing, and displaying data related to patient tissue oxygenation. The system 10 can comprise, for example, several components used for measuring, analyzing, and displaying a patient's tissue oxygen saturation (StO₂) levels, allowing an operator or clinician to monitor in real-time a patient's dynamic tissue oxygenation response characteristics. In the embodiment of FIG. 1, the system 10 includes a blood pressure device 12, a tissue oxygen sensor 14, and a control module 16 in communication with both the blood pressure device 12 and sensor 14.

The blood pressure device 12 includes a pneumatic cuff, tourniquet or other suitable means 18 for restricting blood flow to a selected tissue region of the body. In some embodiments, the blood pressure device 12 is placed on an arm or leg of the patient, and is configured to restrict all or substantially all of the blood flow to a selected tissue region, including both arterial and venous blood flow. In one embodiment, the restriction means 18 may exert up to about 50 mmHg of pressure above the patient's systolic blood pressure in order to entirely or substantially restrict blood flow to the tissue region. In another embodiment, the restriction means 18 may exert at least about 10 mmHg of pressure less than the patient's diastolic blood pressure. In this manner, venous blood flow, but not arterial blood flow, will be entirely or substantially restricted. The restriction means 18 may be manually operated to restrict or permit blood flow or, as discussed further herein, may be automatically controlled by the control module 16.

In the embodiment of FIG. 1, the blood pressure device 12 further includes an integral blood pressure sensor 20 configured to sense the patient's blood pressure immediately prior to the commencement of tissue oxygen measurements from the tissue oxygen sensor 14. In other embodiments, the blood pressure sensor 20 can be integrated into the control module 16, or can comprise a remote device separate from the blood pressure device 12 and control module 16. Blood pressure measurements sensed by the sensor 20 are transmitted to the control module 16, which uses the measurements as feedback to determine whether the restriction means 18 is inflated to a target tourniquet pressure sufficient for stopping blood flow and inducing ischemic conditions in the patient's arm or limb. In some embodiments, for example, the blood pressure measurements can be used by the control module 16 as feedback to control the restriction means 18 during a VOT test, allowing the control module 16 to dynamically adjust the inflation pressure to a target pressure level above the patient's systolic blood pressure. By way of example and not limitation, the control module 16 may use the blood pressure measurements as feedback to maintain the restriction means 18 at a pressure at or about 50 mmHg above systolic pressure.

In other embodiments, blood flow to the selected tissue region may be reduced by controlling the temperature of the selected tissue region. For example, it is known that blood flow may be reduced by lowering tissue temperature and increased by raising tissue temperature. Thus, the restriction means 18 may be a heating or cooling mechanism fitted over a portion of the patient's anatomy. In still other embodiments, blood flow to the selected tissue region may be reduced by raising the selected tissue region higher than the patient's heart or trunk. This may be accomplished by lifting a portion of the patient's anatomy, as, for example, by raising the patient's arm, or by raising a portion of the hospital bed to raise the patient's legs.

The tissue oxygen sensor 14 includes a noninvasive, fiber optic light that illuminates tissue below the level of the skin. The light source for the sensor 14 may be located either in a housing of the sensor 14, or can be located remotely from the sensor 14 (e.g., within the control module 16 or within another device optically coupled to the sensor 14). In one embodiment, the sensor 14 is a near infrared spectroscopy (NIRS) sensor, which uses specific, calibrated wavelengths of near infrared light to noninvasively illuminate a region of tissue below the skin. These wavelengths of light scatter in the tissue and are absorbed differently depending on the amount of oxygen attached to a tissue chromophore (e.g., hemoglobin) in the arterioles, venules, and capillaries. Light that is not absorbed is returned to the sensor 14. The returned light may be transmitted as an optical signal, and can be analyzed to produce a ratio of oxygenated hemoglobin to total hemoglobin, expressed as % StO₂. Tissue chromophore data may also be expressed as tissue oxygenation, tissue deoxygenation and/or total amount of hemoglobin in the tissue. An example NIRS sensor that can be used with the system 10 for sensing tissue chromophore data is described in U.S. Pat. No. 7,596,397, entitled “Patient Interface For Spectroscopy Applications,” which is incorporated herein by reference in its entirety for all purposes.

The tissue oxygen sensor 14 may be placed on any location that is located distal or downstream from the restriction means 18 in relation to arterial blood flow. For example, the sensor 14 may be placed on the thenar muscle of the thumb while the restriction means 18 is located on the upper or lower arm. Alternately, the sensor 14 may be located on the hypothenar, the forearm, the upper arm, the deltoid, the calf, etc., with the restriction means 18 located proximally or upstream of the sensor 14. During a vascular occlusion test, taking the blood pressure measurements on the same arm or limb and using the same cuff that is used for restricting blood flow during the test helps to reduce measurement errors. For example, using the same cuff can reduce measurement errors associated with using one cuff for initially sensing blood pressure and another cuff for later restricting blood flow during a vascular occlusion test.

Since NIRS is capable of measuring localized tissue oxygenation levels, the tissue oxygen sensor 14 may be positioned at a particular area of interest or multiple areas of interest for monitoring and diagnosing shock, sepsis, or other types of diseases as well as monitoring a patient's overall health. For example, the sensor 14 may be placed adjacent to an area of trauma so as to measure tissue oxygenation of the traumatized or healing tissues. The sensor 14 may also be placed over areas where infection is known or suspected to exist. The sensor 14 may also be placed in locations known to be provided with good arterial blood flow or having certain types of tissue which are more easily illuminated by the sensor 14.

The blood pressure device 12, tissue oxygen sensor 14, and control module 16 may be provided with a variety of means of communicating with one another, including both wired and wireless communication modes. In the embodiment of FIG. 1, for example, the blood pressure device 12 and tissue oxygen sensor 14 are connected to the control module 16 via wired electrical and optical connections. In other embodiments, the connections between the blood pressure device 12, sensor 14, and control module 16 are wireless. In one embodiment, the blood pressure device 12 can be configured to communicate wirelessly with the control module 16 (e..g, via RF or inductive telemetry) whereas the tissue oxygen sensor 14 is coupled to the control module 16 via a fiber-optic cable or electrical wires. Other modes of communication are also possible.

The control module 16 is configured to control the operation of the tissue oxygen sensor 14 and to analyze data generated by the sensor 14. In addition, and in some embodiments, the control module 16 is further configured to control the operation of the blood pressure device 12, including the inflation and deflation of the restriction means 18 and analyzing blood pressure measurements taken by the blood pressure sensor 20. A user interface 22 equipped with a monitor 24 can be used to display data obtained from the blood pressure device 12 and tissue oxygen sensor 14, information derived from sensed blood pressure and tissue oxygenation data (e.g., % StO₂, StO₂, THI, systolic blood pressure, diastolic blood pressure, mean blood pressure, pulse rate, etc.), as well as information regarding the operational status of the blood pressure device 12, tissue oxygen sensor 14, and control module 16.

The monitor 24 can be configured to display information relating to the blood pressure sensed by the blood pressure device 12 as well as the tissue oxygenation data sensed by the tissue oxygen sensor 14. The display of such information may take a variety of formats. In some embodiments, for example, the monitor 24 can be configured to display text, graphs or waveforms relating to contemporaneously acquired data, historical data, mean data, or any combination thereof. The monitor 24 can also be used to provide instructions as to the use of the system 10 and to display notices or warnings related to the operation and functionality of the system 10.

The control module 16 and user interface 22 may be integrated into a single unit, as shown in FIG. 1, or may comprise separate components from one another. In some embodiments, the control module 16 may be integrated into an automatic blood-pressure monitoring device such as that commonly found in hospitals or clinics. In one embodiment, the user interface 22 and monitor 24 are embodied in a remote touch screen device that includes a selection pen and graphical user interface that can be used for displaying information and controlling the operation of the blood pressure device 12, tissue oxygen sensor 14, and/or control module 16. Data stored in the touch screen device can be automatically exported to the control module 16 and/or to one or more other memory devices when inserted into a docking station or USB port, or upon the selection of a button or icon on the touch screen. In some embodiments, the various system components form a VOT apparatus that can be used at a remote location, such as at the patient's home.

In use, the remotely used VOT apparatus may permit a patient to take measurements over a period of weeks or months, allowing the patient to gather long-term data showing the development of heart or vascular disease or other ongoing health issues. Data collected by the blood pressure device 12 and tissue oxygen sensor 14 can be stored for later use and can be transmitted to a service that analyzes the data and responds with any changes required to the patient's therapy or monitoring.

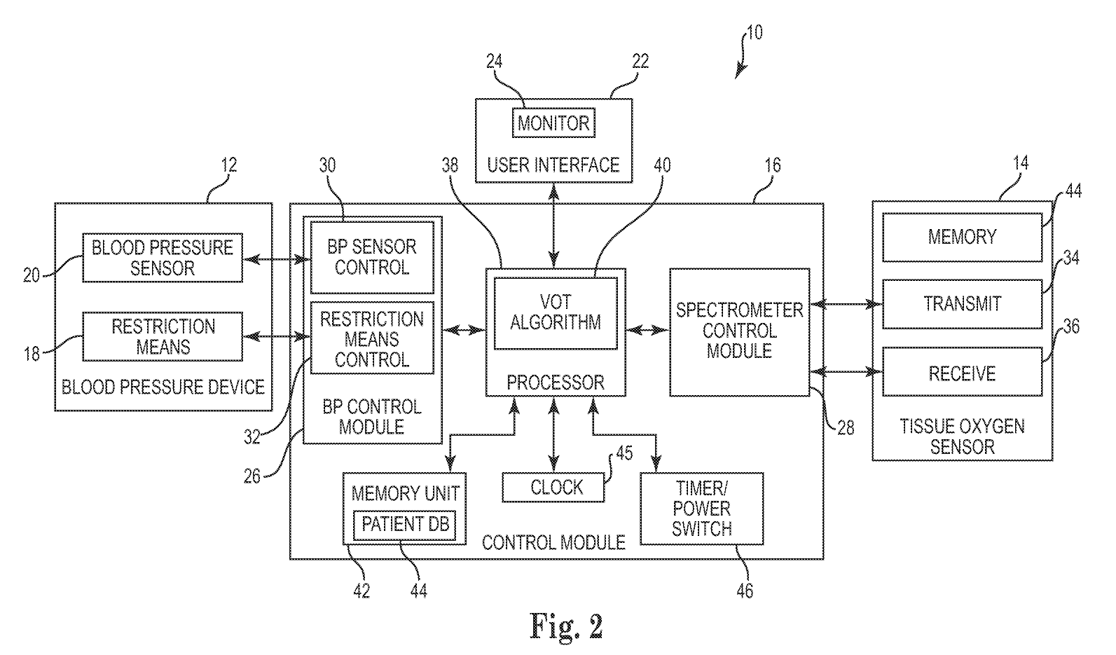

FIG. 2 is a schematic view showing several illustrative components of the blood pressure device 12, tissue oxygen sensor 14, and control module 16 of FIG. 1. In the embodiment of FIG. 2, the control module 16 includes a blood pressure control module 26 and a spectrometer control module 28. The blood pressure control module 26 is configured to control the blood pressure device 12, and includes blood pressure sensor control unit 30 that interfaces with the blood pressure sensor 20 for taking and analyzing blood pressure measurements, and a restriction means control unit 32 that interfaces with the restriction means 18 to provide tourniquet pressure to the patient at selective times before and during a VOT test. The spectrometer control unit 28 is configured to control the transmission of NIRS light to a transmit orifice 34 on the tissue oxygen sensor 14, and receives reflected light back from the patient via a receive orifice 36. The blood pressure and spectrometer control units 26,28 can comprise hardware and/or software within the control module 16. Although separate control units 26,28 are shown in the embodiment of FIG. 2, in other embodiments the control units 26,28 can be integrated together into a single control unit, or can comprise part of other hardware/software within the control module 16.

A computer processor 38 within the control module 16 is configured to perform an algorithm or routine 40 that analyzes information and data obtained from the blood pressure device 12 and tissue oxygen sensor 14, and from this information, determines various characteristics associated with the patient's tissue oxygenation, including % StO₂, tissue hemoglobin index (THI), StO₂ ischemia slope (ΔStO₂)/minute), and StO₂ recovery slope (ΔStO₂/second). The processor 38 is also configured to determine recovery delta THI, which can be defined as the peak total hemoglobin during blood flow recovery minus THI magnitude before or during blood flow restriction. These characteristics can be provided to the user interface 22 and displayed on the monitor 24, allowing the clinician to monitor and diagnose various conditions relating to the patient's health. The measurements can also be stored within a memory unit 42. In some embodiments, the tissue oxygen sensor 14 may also include a dedicated memory unit 44 for storing tissue oxygenation data for later use.

A patient database 44 stored within the memory unit 42 can contain patient information as well as any historical data collected from each patient. Example patient information that can be stored within the database 44 can include, but is not limited to, the patient's name, a patient identifier number, age/date of birth, and gender. The patient database 44 can also contain historical blood pressure and tissue oxygenation data gathered from each patient. Information stored in the patient database 44 can be associated with a unique patient identifier, allowing the patient or patient's clinician to load and display the patient's data on the monitor 24 when entered and recognized by the control module 16.

A real-time clock 45 within the control unit 16 may provide timing signals to the processor 38 and control units 26,28 for use in timing various tasks performed by the blood pressure device 12 and tissue oxygen sensor 14. The clock 45 can also be used for time-stamping measurements obtained from the blood pressure device 12 and tissue oxygen sensor 14 as well as for performing other tasks. As a safety precaution to prevent prolonged cuff inflation, a timer/power switch 46 can be used to monitor the inflation time of the restriction means 18, and can be configured to provide power to the restriction means 18 only when a vascular occlusion test cycle is being performed and for a predetermined period of time. When the timer reaches a certain period of elapsed time (e.g., 10 minutes after cuff inflation), the power to the blood pressure device 12 can be switched off via the timer/power switch 46, forcing the restriction means 18 to deflate. In some cases, this hardware feature can help prevent an unintentional prolonged ischemia time in the event of a software and/or communication failure within the control module 16 or within the blood pressure device 12.

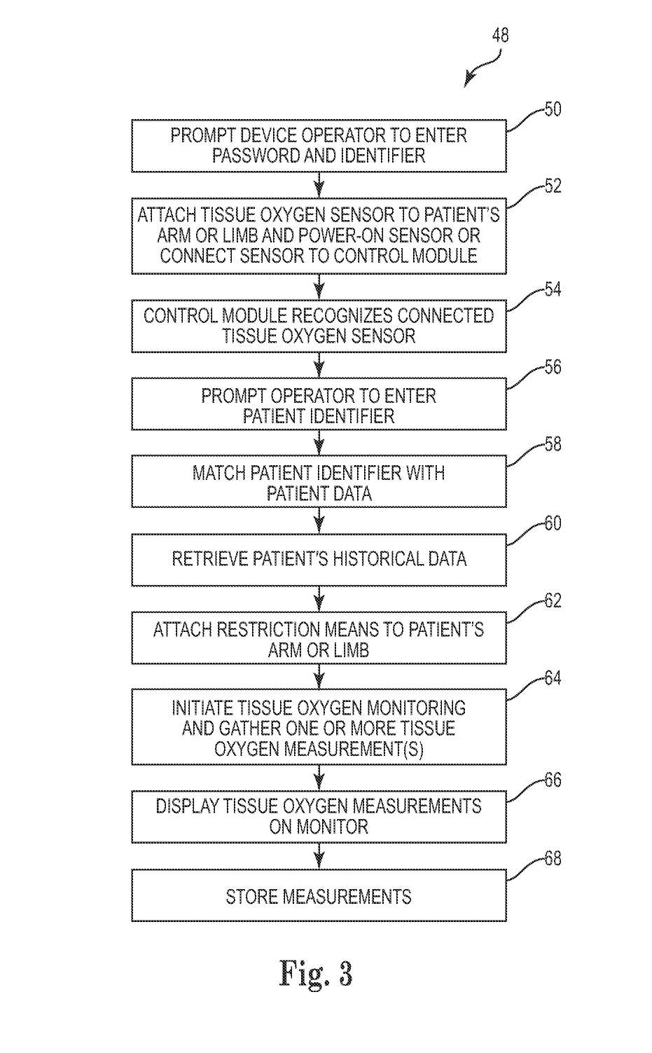

FIG. 3 is a block diagram showing an example process 48 for configuring the blood pressure device 12, tissue oxygen sensor 14, and control module 16 of FIGS. 1-2 for use with a particular patient for taking tissue oxygen measurements. The process 48 may begin generally at block 50, in which the control module 16 prompts the device operator to enter a login password and identifier (e.g., the operator name or initials) associated with the device operator. The device operator login password and identifier is used to satisfy compliance with controlling access to patient records or data, and to provide a history of which operator performed a particular test on the patient. The login password and identifier also serves to restrict use of the apparatus to trained and/or pre-authorized users.

The control module 16 may prompt the operator to attach the tissue oxygen sensor 14 to the patient's arm or limb and to the control module 16, and then power-on the sensor 14 or connect the sensor 14 to the control module 16 (block 52). Upon power-up, the control module 16 can be configured to automatically recognize the connected sensor 14 (block 54) and, once recognized, prompt the operator to enter a patient identifier identifying the particular patient to undergo tissue oxygenation monitoring or a VOT test (block 56). The patient identifier may comprise, for example, the last name of the patient or an identification number associated with the patient contained in the patient database 44. In some embodiments, the patient identifier can be inputted to the control module 16 via a bar code scanner or stylus pen provided as part of a remote user interface/monitor. Once the patient identifier has been entered and matched with the patient data contained in the patient database 44 (block 58), the control module 16 can be configured to automatically retrieve the patient's historical data for viewing and/or exporting (block 60), thus providing the operator with easy access to this information during VOT testing.

Once a patient identifier is entered and configured for use with the patient, and in some embodiments, the control module 16 may then prompt the operator to place the restriction means 18 on either the upper or lower portion of the same arm or limb where the tissue oxygen sensor 14 is placed (block 62). In other embodiments, the control module 16 may prompt the operator to place the restriction means 18 on the patient at a different time during the process 48, such as immediately before or after attaching the tissue oxygen sensor 14 to the patient. Once attached, the operator can then initiate tissue oxygen monitoring via the user interface and gather one or more tissue oxygenation measurements (block 64). The control module 16 may then display the tissue oxygen measurements as well as other measured parameters on the monitor 24 for that patient (block 66). The control module 16 can also be configured to periodically store such measurements for further analysis (block 68). In certain embodiments, for example, the measured parameters can be stored within the memory unit 42 of the control module 16 and/or transmitted to another device for storage.

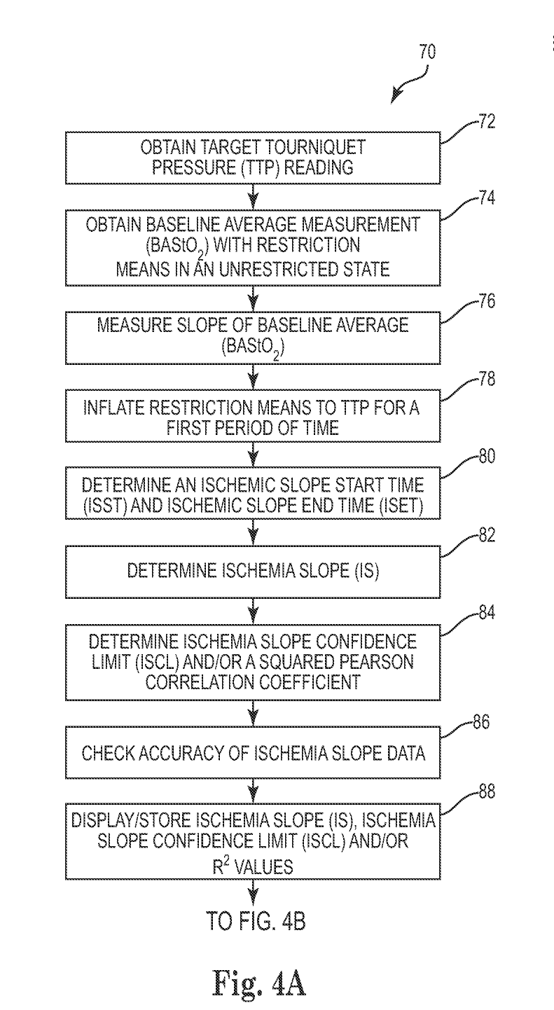

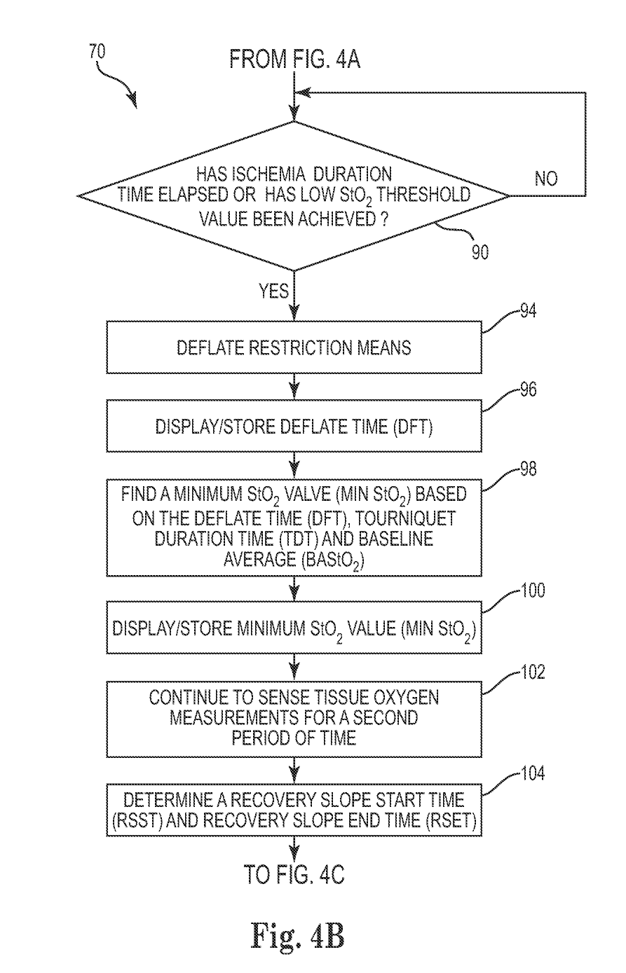

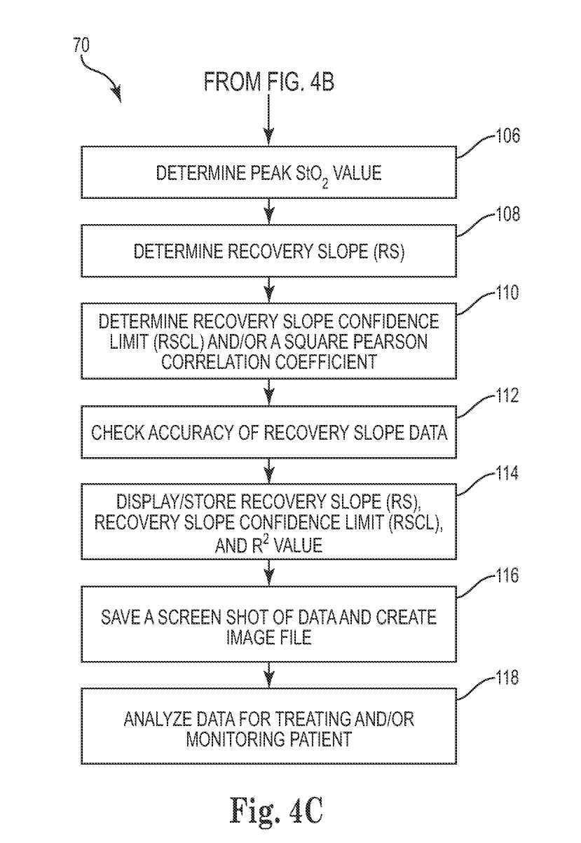

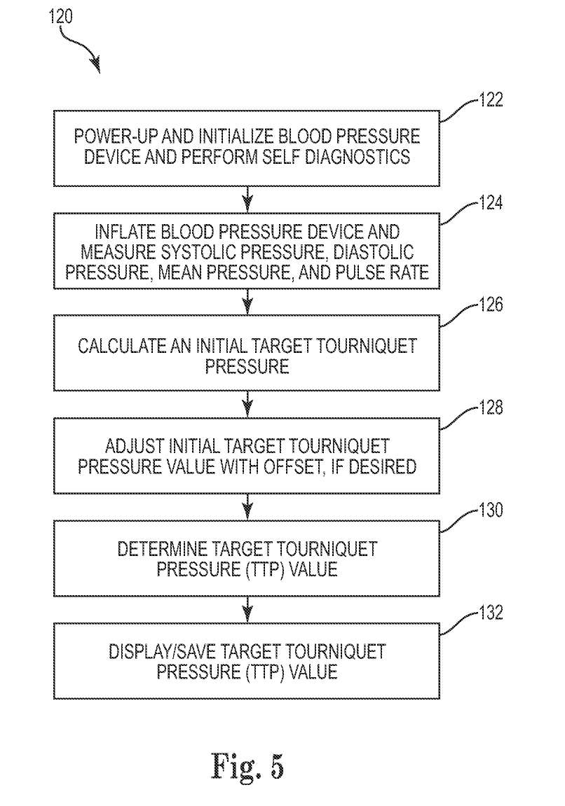

FIGS. 4A-4C is a block diagram showing an example process 70 for obtaining one or more tissue oxygen measurements from a patient. The process 70 may represent, for example, several example steps used by the algorithm or routine 40 of FIG. 2 to analyze tissue oxygen measurements taken with the tissue oxygen sensor 14. As shown in FIG. 4A, the process 70 may begin generally at block 72, in which a target tourniquet pressure (TTP) reading is obtained at a time immediately prior to performing a VOT test on the patient. In certain embodiments, for example, the TTP reading can be obtained using the same blood pressure device 12 that is later used for restricting blood flow to the patient's arm or limb during a VOT test. The TTP reading represents the pressure needed to stop blood flow to the measurement site, and is independent of the cuff design, the placement location relative to the tissue oxygen sensor 14, and/or the patient's posture or orientation. An example target tourniquet pressure range can comprise 50 mmHg to 300 mmHg, with increments of ±1 mmHg. Several example steps that can be used for determining a TTP reading are further described herein with respect to FIG. 5.

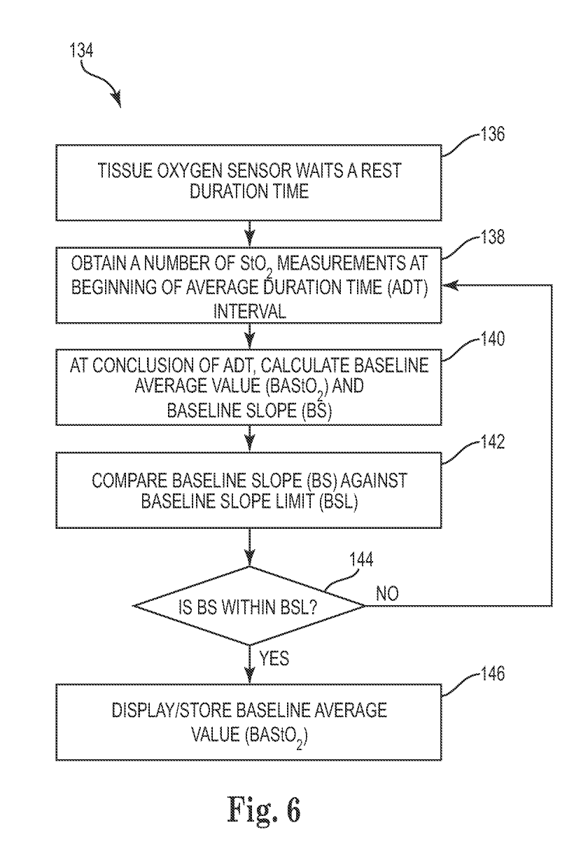

Once a TTP reading is obtained, the restriction means 18 used to identify the TTP reading can be deflated and a baseline average measurement is taken by the tissue oxygen sensor 14 in an unrestricted state in which blood flow is not restricted to the selected region (block 74). The baseline average measurement can be used by the control module 16 to define an ischemia slope start time and a recovery slope end time measured later during a VOT test, and can be expressed as a baseline average value (BAStO₂) on the monitor 24. In some embodiments, the BAStO₂ measurement can be used by the control module 16 to monitor baseline stability and to improve baseline calculation accuracy, which can affect the measurements made during later steps in the testing process. Several example steps that can be used for determining a BaStO₂ value are further described herein with respect to FIG. 6.

The control module 16 can then be configured to measure a slope of BAStO₂ value (block 76). If the slope of the BAStO2 value is relatively stable (e.g., at or near zero), then a VOT test may then be performed by inflating the restriction means 18 to the previous calculated TTP value obtained from the blood pressure sensor 20 to establish ischemia conditions at the measurement site during a first period of time (block 78). The sensing of StO₂ measurements can be taken at fixed intervals during this period, such as every 2 seconds. The time at which the restriction means 18 is first activated or inflated to TTP can be stored in the memory unit 42 and displayed on the monitor 24 for evaluation by the clinician.

Upon inflating the restriction means 18 to establish ischemic conditions, the control module 16 may next determine an ischemia slope start time (ISST) and an ischemia slope end time (ISET) associated with the ischemia (block 78). In certain embodiments, a tissue oxygenation change threshold (e.g., 95% of baseline reading) may be applied to the previously calculated baseline average value to determine the ISST:

ISST=first time when StO₂≦0.95(BAStO₂). (1)

The tissue oxygen change threshold is used to find an ISST where StO₂ begins to decrease with time, and can be any value between 0 and 1, and more typically, between 0.5 and 1.

To determine an ischemia slope end time (ISET) associated with the ischemia, and as expressed in equation (2) below, the control module 16 may then add the ISST value to an ischemia slope duration time (ISDT) corresponding to a time interval that is less than or equal to the total duration time of the induced ischemia:

ISET=ISST+ISDT. (2)

The ISDT can be preconfigured to a known value that generally represents the first linear region of the tissue oxygenation decay with ischemia, or can be automatically chosen by the control module 16 to best represent the constant slope region of the tissue oxygen measurements during ischemia. The first constant slope (i.e., linear) region of tissue oxygenation decay during ischemia is believed to represent the metabolic activity or oxygen consumption rate prior to inducing cuff ischemia. During cuff ischemia, the tissue oxygen decay slope may deviate from a linear shape or constant value as the ischemia time progresses and regional oxygen delivery or flux begins to limit oxygen consumption. The ISET may also be chosen to match the inflection point where the ischemia slope first begins to deviate from a linear or constant value.

From the ISST and ISET values, an ischemia slope (IS) may then be determined by the control module (16) (block 82). In certain embodiments, the ischemia slope may be determined by calculating the slope (m) of the following linear equation:

Yᵢ=mXᵢ+B; (3)

where:

Yᵢ are the measured hemoglobin oxygen saturation (StO₂) values between the ISST and the ISET; and

Xᵢ are the paired times between the ISST and the ISET.

In some embodiments, multiple StO₂ measurements can be used to calculate an average StO₂ measurement value for use in determining the ischemia slope. In one embodiment, for example, the control module 16 may perform a block average of five consecutive StO₂ measurements in order to obtain an average StO₂ measurement value over a period of time. To ensure reliability, three valid readings of the individual StO₂ values may be required to produce a valid average StO₂ measurement. Second order polynomial smoothing can also be applied to the StO₂ measurements to produce a smoother function when displayed on the monitor 24.

The determination of slope (m) for each individual hemoglobin oxygen saturation (StO₂) data point Yᵢ at time Xᵢ can also be determined based on the following equation:

$$m = \frac{{\sum }_{ISST}^{ISET} ( Xi - Xavg ) ( Yi - Yavg )}{{\sum }_{ISST}^{ISET} {( Xi - Xavg )}^{2}} ; \qquad ( 4 )$$

where:

Yavg is an average hemoglobin oxygen saturation between the ISST and the ISET; and

Xavg is an average time between the ISST and the ISET.

From the slope (m) calculation above, the control module 16 may then calculate an ischemia slope confidence limit (ISCL) value and/or a squared Pearson correlation coefficient (R²) using the StO₂ data points between the ISST and the ISET (block 84). The ISCL value represents the calculated ischemia slope's 95% confidence interval limits, and describes the accuracy of the slope measurement. The measured accuracy can then be used to assess whether equation (3) used to fit the data provides a good degree of fit and that the measured slope is trustworthy for influencing a treatment decision or therapy action. If the degree of fit is poor, the operator may check for the proper position and location of the tissue oxygen sensor (14), restriction means (18), as well as the posture of the patient, and then decide whether to replicate the measurement. In some embodiments, the ISCL value can be determined based on the following equation:

ISCL=m±tcritical\sqrt{m_{variance}}; (5)

where:

$${m}_{variance} = \frac{SSE / n - 2}{{\sum }_{ISST}^{ISET} {( Xi - Xavg )}^{2}} ;$$

$$tcritical = 1.949145 + 2.78035 / ( n - 2 ) - 0.13860459 / {( n - 2 )}^{2} + 8.114116 / {( n - 2 )}^{3} ;$$

$$SSE = {\sum }_{ISST}^{ISET} {Yi}^{2} - B {\sum }_{ISST}^{ISET} Yi - m {\sum }_{ISST}^{ISET} XiYi ;$$

B is an offset valued determined by:

$$\frac{{\sum }_{ISST}^{ISET} Yi - m {\sum }_{ISST}^{ISET} Xi}{n} ;$$

and

n is the number of measurements.

In some embodiments, the tcritical equation used in determining ISCL is a polynomial equation fit to a Student's t distribution. In other embodiments, a lookup table or other forms of equations may also be used to compute tcritical.

The squared Pearson correlation coefficient (R²) is another statistic that can be used to assess the degree of fit of equation (3) above. In some embodiments, the squared Pearson correlation coefficient (R²) can be determined based on the following equation:

R²=1−SSE/SST; (6)

where:

$$SSE = {\sum }_{ISST}^{ISET} {Yi}^{2} - B {\sum }_{ISST}^{ISET} Yi - m {\sum }_{ISST}^{ISET} XiYi ; and$$

$$SST = {\sum }_{ISST}^{ISET} {Y}^{2} - \frac{{( {\sum }_{ISST}^{ISET} Yi )}^{2}}{n} .$$

As the magnitude of the calculated slope from equation (3) above changes or approaches zero, the R² value will also change or approach zero regardless of whether the degree of fit remains accurate. Thus, the R² value does not provide a unique threshold value for assessing accuracy, or degree of fit, for all possible slope magnitudes. The confidence interval for the slope (e.g., 95% in equation (5) above) does not depend on the slope magnitude, and better represents the accuracy, or degree of fit, of a slope measurement.

The control module 16 can be configured to check the fitness of the ischemia slope data by comparing the measured ischemia slope 95% confidence limit (ISCL) against an ischemia slope confidence interval acceptance limit to determine if a calculated slope is usable or accurate enough to affect a decision or therapy (block 86). The ischemia slope confidence interval acceptance limit can be preprogrammed to a default value that can be adjusted by the operator or a technician, if desired. In some embodiments, the control module 16 can be configured to require a minimum number of measured data points between the ISST and ISET to ensure that the slope calculation in equation (3) above is sufficiently reliable. In some embodiments, the data fitness check can be performed by determining whether there are at least 25 valid data points between the ischemia slope acceptance limit and the ischemia slope confidence limit. At the conclusion of the ischemia slope determination, the ischemia slope, ischemia slope confidence limits, and/or R² values can be displayed and stored (block 88). A message may also be displayed on the monitor 24 informing the operator whether the ischemia slope is within acceptable limits, and is thus usable.

Once the ischemia slope is determined and confirmed to be accurate, the control module 16 may then wait until a certain ischemia duration time has elapsed or until a low StO₂ threshold value has been achieved (block 90). In some embodiments, the control module 16 may determine whether to deflate the restriction means 18 if the inflation time is at or greater than the time when the restriction means 18 is first inflated plus a tourniquet duration time (TDT) programmed within the control module 16. Alternatively, or in addition, the control module 16 may determine whether to deflate the restriction means 18 based on whether a measured StO₂ value is at or less than a low StO₂ limit programmed within the control module 16. If either one of these conditions are satisfied, the control module 16 may then deflate the restriction means 18 (block 94), reestablishing blood flow to the selected tissue region. The control module can store/display a deflate time (DFT) value representing the time at which the restriction means 18 begins to deflate (block 96).

The control module 16 can then be configured to find a minimum StO₂ value (MinStO₂) based on the deflate time (DFT), the tourniquet duration time (TDT), and the baseline average value (BAStO₂) (block 98). In some embodiments, the control module 16 may find the lowest measured StO₂ value starting at the deflate time (DFT) and ending at the time when an StO₂ value exceeds the BAStO₂ or whether the time exceeds the deflate time plus a predetermined time interval (e.g., 1 minute). The minimum StO₂ value and the time associated with that value can then be displayed and stored (block 100).

As the restriction means 18 is deflated to re-establish blood flow to the measurement region, the tissue oxygen sensor 14 continues to collect tissue oxygen data over a second time period associated with blood flow recovery (block 102). During the recovery time period, the sensing of StO₂ measurements can be taken at fixed intervals equal to or faster than the update rate during the ischemia interval. In some embodiments, for example, the recovery StO₂ update rate may be a time at or less than about 2 seconds, such as 400 ms. Since the time interval for which a recovery slope is calculated can be significantly less than the time interval for which an ischemia slope is calculated, a faster measurement update rate may be necessary during the recovery phase to ensure that there are a sufficient number of data points to reliably calculate the recovery slope.

Using the minimum StO₂ value (MinStO₂) and minimum time value (MinTime), the control module 16 may next determine a recovery slope start time (RSST) and recovery slope end time (RSET) (block 104). As with the determination of the ischemia slope start time and ischemia slope end time, a tissue oxygenation change threshold (i.e., 102% of minimum StO₂ reading) may be used in determining the RSST and RSET values. In some embodiments, for example, the RSST value may be determined based on the first time after (MinTime) in which StO₂ is at or greater than the minimum StO₂ value (MinStO₂) by a percentage of the minimum StO₂ value, as set forth in the following equation:

RSST=first time after MinTime when StO₂≧1.02(MinStO₂). (7)

The “1.02” value in equation (7) above defines when StO₂ has increased by a given percentage, and can be any value between 1 and 2, and more typically, between 1 and 1.5.

The recovery slope end time (RSET), in turn, can be determined based on the following equation:

RSET=the first time after MinTime when (StO₂−MinStO₂)≧BRF(BAStO₂−MinStO₂); (8)

where BRF is a baseline recovery fraction programmed within the control module 16 and describes the percentage of change between the baseline average (BAStO₂) and MinStO₂. BRF can be a value between 0 and 1, and is typically chosen to be in the range of 0.5 to 1. A BRF value of 0.85, for example, may ensure that the recovery slope mostly includes the first linear region where StO₂ increases with time.

Similar to ISET, RSET can be preconfigured to a known value that generally approximates the first linear region of the tissue oxygenation increase with recovery, or can be automatically chosen by the control module 16 to best represent the constant slope region of the tissue oxygenation measurement during recovery. The first constant slope (i.e., linear) region of tissue oxygenation recovery may represent the rate of regional blood flow or oxygen reperfusion immediately after restoring blood flow. The RSET may also be chosen to match the inflection point where the recovery slope first begins to deviate from a linear or constant value.

From the recovery slope end time (RSET), a peak StO₂ value corresponding to the maximum StO₂ value occurring between the MinTime and (MinTime+TDT) can be determined (block 106). If the StO₂ value does not recover to baseline during this time period, then the RSET value can be determined based on the following equation:

RSET=the first time after MinTime when (StO₂−MinStO₂)≧BRF(PeakStO₂−MinStO₂). (9)

Once the RSST and RSET are determined, a recovery slope (RS) may then be determined (block 108). In certain embodiments, the RS may be determined by calculating the slope (m) of the following linear equation:

Yᵢ=mXᵢ+B; (10)

where:

Yᵢ are the measured hemoglobin oxygen saturation (StO₂) values between the RSST and the RSET; and

Xᵢ are the paired times between the RSST and the RSET.

As with the StO₂ measurements taken during the first period of time, multiple StO₂ measurements can be used to calculate an average StO₂ measurement value for use in determining the recovery slope. In one embodiment, for example, the control module 16 may perform a block average of five consecutive StO₂ measurements in order to obtain an average StO₂ measurement value over a period of time. To ensure reliability, three valid readings of the individual StO₂ values may be required to produce a valid average StO₂ measurement.

The determination of slope (m) for each individual hemoglobin oxygen saturation (StO₂) data point Yᵢ at time Xᵢ can also be determined based on the following equation:

$$m = \frac{{\sum }_{IRST}^{RSET} ( Xi - Xavg ) ( Yi - Yavg )}{{\sum }_{RSST}^{RSET} {( Xi - Xavg )}^{2}} ; \qquad ( 11 )$$

where:

Yavg is the average StO2 between the RSST and the RSET; and

Xavg is the average time between the RSST and the RSET.

From the slope (m) calculation above, the control module 16 may then calculate a recovery slope confidence limit (RSCL) value and/or a squared Pearson correlation coefficient (R²) using the StO₂ data points between the RSST and the RSET (block 110). The RSCL value is associated with the calculated recovery slope's confidence interval, and represents a value of the accuracy of the recovery slope measurement for determining whether the recovery slope calculation is accurate and trustworthy for influencing a treatment or therapy action. In some embodiments, the RSCL value can be determined based on the following equation:

RSCL=m±tcritical\sqrt{m_{variance}}; (12)

where:

$$\frac{{\sum }_{RSST}^{RSET} Yi - m {\sum }_{RSST}^{RSET} Xi}{n} ;$$

B is an offset valued determined by:

$$SSE = {\sum }_{RSST}^{RSET} {Yi}^{2} - B {\sum }_{RSST}^{RSET} Yi - m {\sum }_{RSST}^{RSET} XiYi ; and$$

$$SST = {\sum }_{RSST}^{RSET} {Y}^{2} - \frac{{( {\sum }_{RSST}^{RSET} Yi )}^{2}}{n} .$$

and

n is the number of measurements.

In some embodiments, the tcritical equation used in determining RSCL is a polynomial equation fit to a Student's t distribution. In other embodiments, a lookup table or other forms of equations may also be used to compute tcritical.

A squared Pearson correlation coefficient (R²) can also be used as another statistic to describe and assess the degree of fit of the recovery slope. In some embodiments, the squared Pearson correlation coefficient (R²) can be determined based on the following equation:

R²=1−SSE/SST; (13)

where:

$${m}_{variance} = \frac{SSE / n - 2}{{\sum }_{RSST}^{RSET} {( Xi - Xavg )}^{2}} ;$$

$$tcritical = 1.949145 + 2.78035 / ( n - 2 ) - 0.13860459 / {( n - 2 )}^{2} + 8.114116 / {( n - 2 )}^{3} ;$$

$$SSE = {\sum }_{RSST}^{RSET} {Yi}^{2} - B {\sum }_{RSST}^{RSET} Yi - m {\sum }_{RSST}^{RSET} XiYi ;$$

The control module 16 can be configured to check the fitness of the recovery slope data by comparing the measured recovery slope 95% confidence limit (RSCL) against a recovery slope confidence interval acceptance limit (RSAL) and a minimum number of measurement points between RSST and RSET, and from this data, determine whether the slope is sufficiently accurate or trustworthy (block 112). In some embodiments, the data fitness check can be performed by determining whether there are at least 10 data points between the RSST and the RSET. At the conclusion of the recovery slope determination, the recovery slope, recovery slope confidence limits, and R² values can be displayed and stored (block 114). A message may also be displayed on the monitor 24 informing the operator whether the recovery slope is within range, and is thus usable.

The control module 16 may then wait for a period of time to allow the tissue oxygenation to fully recover from the ischemia event, and then save a screenshot and image file of the data (block 116). In those embodiments in which a graphical representation of the StO₂ data is displayed on a monitor 24, the algorithm or routine 40 may automatically scale the graph x axis such that all of the data taken during each stage of the VOT test appears on the monitor. If desired, the clinician may then analyze the data for treating or monitoring the patient (block 118). To assist the clinician in accurately judging the accuracy of the data, the monitor 24 can be configured to display fitted slope lines and interval points along with captions. Visual indicators on the monitor 24 can show roughly linear segments of baseline average, pre-test stability, ischemia slope, and recovery slope. Information specific to the patient can also be provided along with the data and visual effects on the monitor 24.

The various computed values, including the baseline average StO₂, ischemia slope and recovery slope, blood pressure and/or pulse rate can be used to monitor and characterize a patient's physiologic state based on their ischemic response in relation to a control response of a known, control population. An example system for characterizing tissue chromophore data and then comparing this data against characterizing data from a control population is further described herein with respect to U.S. Pat. No. 7,536,214, entitled “Dynamic StO2 Measurements and Analysis,” which is incorporated herein by reference in its entirety for all purposes.

FIG. 5 is a block diagram showing an example process 120 for obtaining a target tourniquet pressure (TTP) reading using the system 10 of FIG. 1. The process 120 may represent, for example, several illustrative steps of block 72 in FIG. 4A. As shown in FIG. 5, the process 120 may begin at block 122, in which the blood pressure device 12 is initially powered on and activated for a period time sufficient for the device 12 to perform various self-diagnostics and initialization routines. After this initialization period, the blood pressure device 12 is then inflated and the blood pressure sensor 20 is tasked to measure the patient's systolic blood pressure, diastolic blood pressure, mean blood pressure, and pulse rate (block 124).

Once the patient's systolic blood pressure is measured, the control module 16 may next calculate an initial target tourniquet pressure (TTPᵢ) that can be later used to inflate the restriction means 18 to a sufficient pressure during a later VOT test (block 126). An upper blood pressure limit such as 245 mmHg can be used as a starting point for measuring the patient's systolic blood pressure. In some embodiments, the initial target tourniquet pressure (TTPᵢ) value can be obtained by adding an offset value (e.g., 50 mmHg) to the patient's systolic blood pressure value (SBP), as shown in the following equation:

TTPᵢ=SBP+50 mmHg. (14)

An offset value (ΔTTP) can also be provided to adjust the initial target tourniquet pressure value (TTPᵢ) by a specified amount, if desired (block 128). The offset value (ΔTTP) can be inputted, for example, by an operator or clinician to compensate for the particular type of restriction means 18 used or if the blood pressure module has difficulty in automatically determining TTP. In those embodiments in which an offset value is provided, the target tourniquet pressure (TTP) value can then be determined at block 130 based on the following equation:

TTP=TTPᵢ+ΔTTP. (15)

The target tourniquet pressure (TTP) value can then be displayed and saved (block 132) for later use by the control module 16 in controlling the restriction means 18, and for computing other values such as the baseline average StO₂ value.

FIG. 6 is a block diagram showing an example process 134 for determining a baseline average value (BaStO₂) using the system 10 of FIG. 1. The process 134 may represent, for example, several illustrative steps of block 74 in FIG. 4A. As shown in FIG. 6, the process 134 may begin at block 136, in which the tissue oxygen sensor 14 waits for a rest duration time (RTD), allowing the patient's vitals or peripheral blood flow or oxygen consumption to achieve a steady-state condition. After the rest duration time, and at the beginning of an average duration time (ATD) interval, the tissue oxygen sensor 14 is tasked to obtain a number of StO₂ measurements (block 138).

At the conclusion of the average duration time (ATD) interval, the control module 16 calculates a baseline average and a baseline slope between a first StO₂ value and a last StO₂ value during the ATD (block 140). The baseline slope is then compared against a baseline slope limit (BSL) (block 142) programmed within the control module 16. A check can then be made to determine whether the baseline slope obtained during the current average duration time (ATD) period is between the baseline slope limit (block 144). If not, the average duration time (ATD) period is reset, and up to two more attempts are made to determine the baseline average StO₂ and baseline slope. Otherwise, if the current baseline slope is between the baseline slope limit, then the current baseline average StO₂ value is displayed and stored (block 146).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method for analyzing data related to tissue oxygenation in a patient, comprising:
activating a means for restricting blood flow to an arm or limb of a patient;
determining a target tourniquet pressure for inducing ischemia within the arm or limb;
obtaining a number of baseline tissue oxygen measurements from the patient while the restriction means is in an unrestricted state;
determining a baseline average StO₂ value from the baseline tissue oxygen measurements;
controlling the restriction means to a pressure at or near the target tourniquet pressure during a first period of time;
determining an ischemia slope start time and an ischemia slope end time during the first period of time;
determining an ischemia slope between the ischemia slope start time and the ischemia slope end time;
controlling the operation of the restriction means to un-restrict blood flow to the arm or limb during a second period of time;
determining a recovery slope start time and a recovery slope end time during the second period of time;
determining a recovery slope between the recovery slope start time and the recovery slope end time; and
storing one or more tissue oxygen measurements in a memory unit.

2. The method of claim 1, wherein determining a target tourniquet pressure for inducing ischemia within the arm or limb of the patient comprises:
measuring the patient's systolic blood pressure; and
determining a target tourniquet pressure at or above the systolic blood pressure.

3. The method of claim 2, further comprising adjusting the target tourniquet pressure by an offset pressure value.

4. The method of claim 1, wherein determining a baseline average StO₂ value from the baseline tissue oxygen measurements comprises:
controlling the operation of the restriction means to un-restrict blood flow;
sensing a number of StO₂ measurements;
determining a baseline slope between a first % StO₂ value and a last % StO₂ value during an average duration time interval; and
comparing the baseline slope to a baseline slope limit value to determine if the slope is within an acceptable range.

5. The method of claim 1, wherein the ischemia slope start time and ischemia slope end time is determined based at least in part from the baseline average StO₂ value.

6. The method of claim 5, wherein determining an ischemia slope start time and an ischemia slope end time includes multiplying a fractional change to the baseline average StO₂ value.

7. The method of claim 1, wherein determining an ischemia slope between the ischemia slope start time and the ischemia slope end time includes computing an average StO₂ measurement from a number of individual StO₂ measurements during the first period of time.

8. The method of claim 1, further comprising confirming the accuracy of the ischemia slope.

9. The method of claim 8, wherein confirming the accuracy of the ischemia slope comprises:
determining an ischemia slope confidence limit using % StO₂ data obtained between the ischemia slope start time and the ischemia slope end time; and
comparing the ischemia slope confidence limit against a reference ischemia slope acceptance limit.

10. The method of claim 1, wherein determining a recovery slope start time and a recovery slope end time includes multiplying a fraction change to a minimum StO₂ value from tissue oxygen measurements obtained during the second period of time.

11. The method of claim 1, wherein determining a recovery slope between the recovery slope start time and recovery slope end time includes computing an average StO₂ measurement from a number of individual StO₂ measurements during the second period of time.

12. The method of claim 1, further comprising confirming the accuracy of the recovery slope.

13. The method of claim 12, wherein confirming the accuracy of the recovery slope comprises:
determining a recovery slope confidence limit using % StO₂ data obtained between the recovery slope start time and the recovery slope end time; and
comparing the recovery slope confidence limit against a reference recovery slope acceptance limit.

14. A system for analyzing data related to tissue oxygenation in a patient, the system comprising:
a blood pressure device including a blood pressure sensor and a means for restricting blood flow to an arm or limb of a patient;
a tissue oxygen sensor configured to gather data on a tissue chromophore whose light properties depend on the oxygenated state of tissue;
a control module in communication with the blood pressure device and the tissue oxygen sensor, the control module configured to control the operation of the restriction means based at least in part on one or more measurements sensed by the tissue oxygen sensor; and
a user interface adapted to display blood pressure measurements and tissue oxygen measurements.

15. The system of claim 14, wherein the control module comprises:
a blood pressure control unit configured for controlling the blood pressure device;
a spectrometer control unit configured for controlling the tissue oxygen sensor; and
a processor configured to analyze measurements from the blood pressure device and tissue oxygen sensor.

16. The system of claim 14, wherein control module is configured to control the restriction means during a vascular occlusion test using feedback from the blood pressure sensor.

17. The system of claim 14, wherein the control module includes a memory unit configured for storing a patient database.

18. The system of claim 17, wherein, upon connection of the tissue oxygen sensor to the control module, the control module is configured to prompt a user to identify a patient in the patient database via the user interface.

19. A vascular occlusion test apparatus, comprising:
a blood pressure control module configured for controlling a blood pressure device;
a spectrometer control module configured for controlling a tissue oxygen sensor; and
a processor configured to run an algorithm or routine for analyzing blood pressure measurements and tissue oxygen measurements.

20. The apparatus of claim 19, wherein the processor is adapted to determine a target tourniquet pressure value from the blood pressure and tissue oxygen measurements.
